(19) United States
(12) Patent Application Publication
(10) Pub. No.: US 2025/0338873 A1
(43) Pub. Date: Nov. 6, 2025

(54) PREPARATION OF DUAL CROSS-LINKED ZEIN-CARBOXYMETHYL CHITOSAN NANOPARTICLES FOR IMPROVING THERMAL STABILITY OF POLYPHENOL

(71) Applicant: Jiangnan University, Wuxi (CN)

(72) Inventors: Long CHEN, Wuxi (CN); Wen XU, Wuxi (CN); Hao CHENG, Wuxi (CN); Kai MA, Wuxi (CN); Kuang HE, Wuxi (CN); Zhengyu JIN, Wuxi (CN); Linjun ZHANG, Wuxi (CN); Hongjian CHEN, Wuxi (CN); Hangyan JI, Wuxi (CN); Jianwei ZHAO, Wuxi (CN); Zhengjun XIE, Wuxi (CN)

(21) Appl. No.: 19/269,070

(22) Filed: Jul. 15, 2025

(30) Foreign Application Priority Data
Jul. 22, 2024 (CN) 2024109792917

(51) Int. Cl. A23J 3/14; A23L 29/00; A23L 29/275

(52) U.S. Cl. CPC A23J 3/14; A23L 29/035; A23L 29/275

(57) ABSTRACT

Disclosed is preparation of dual cross-linked zein-carboxymethyl chitosan nanoparticles for improving the thermal stability of polyphenol. After covalently cross-linked zein, tannic acid is used to prepare tannic acid cross-linked zein-carboxymethyl chitosan nanoparticles loaded with quercetin, Ca²⁺ is then added to increase a degree of crosslinking between the tannic acid and the carboxymethyl chitosan, as well as between molecules of the carboxymethyl chitosan in the zein nanoparticles to make the structure tighter, such that structural stability of the nanoparticles can be maintained during the thermal processing after the nanoparticles are formed, the quercetin encapsulated inside is protected well, and a retention rate of quercetin during the thermal processing is improved. The method provided in the present disclosure is simple, green, pollution-free and low energy consumption, and the prepared nanoparticles can improve the thermal stability of quercetin, and can be used as a natural additive for thermal processing of food.

### TECHNICAL FIELD

The present disclosure belongs to the technical field of food additives, and particularly relates to preparation of dual cross-linked zein-carboxymethyl chitosan nanoparticles for improving the thermal stability of polyphenol.

### BACKGROUND

Quercetin is a common flavonol compound with antioxidant, antibacterial, anti-inflammatory, anti-allergic, blood pressure lowering, blood lipid lowering, and immune modulation functions. However, quercetin has low bioavailability, poor water solubility, and extreme instability under light and heat conditions, which significantly limit its use in food thermal processing. The most commonly used thermal processing methods in food production include steaming, blanching, hot extrusion, thermal sterilization, and the like. Thermal treatment is one of the most important methods for improving quality and extending shelf life of the food. Therefore, it is necessary to design a colloidal delivery system to protect thermal stability of the quercetin in steamed food, so as to improve a retention rate of the quercetin in high-temperature environments.

However, most of the colloidal delivery systems available at present are designed based on a gastrointestinal delivery in the human body, and are unsuitable for food thermal processing. A colloidal delivery system is one of the most commonly used methods to improve the stability of polyphenolic substance. It is of great guiding significance to cross-link raw materials for preparing the colloidal delivery system, to improve the thermal stability of the colloidal delivery system in food thermal processing. At present, common cross-linking methods include physical cross-linking, chemical cross-linking and enzymatic cross-linking. A structure of components such as proteins and polysaccharides can be altered or modified through cross-linking, so as to improve their performance, and finally expand an application range of the colloidal delivery system and improve food quality. However, the interaction generated by only one cross-linking method is weak and easily destroyed. Enzymes are prone to inactivation during an enzymatic cross-linking process, and the process is complex and costly.

Currently, a polysaccharide coating is often added to improve the stability of the colloidal delivery system. However, the polysaccharide coating is easily separated from a core part during heating, leading to the escape of encapsulated substance and a loss of activity due to heating. There is a lack of modifications to raw material to improve the application of the colloidal delivery system in steamed and cooked foods, particularly research on dual cross-linking of multiple components of the colloidal delivery system.

### SUMMARY

In order to solve the above problems, the present disclosure adopts a method of dual cross-linking of tannic acid (TA) and Ca²⁺ to modify structures of protein and polysaccharide. Specifically, zein is cross-linked and modified with tannic acid first to prepare a covalent complex, such that protein molecules are more tightly bound, and quercetin molecules are prevented from escaping during cooking. Phenolic hydroxyl groups grafted with the tannic acid in zein-tannic acid nanoparticles loaded with quercetin at pH 6 carry negative charges. Ca²⁺ serves as a salt bridge to connect carboxyl groups of polyampholyte carboxymethyl chitosan and phenolic hydroxyl groups of tannic acid, such that more carboxymethyl chitosan is adsorbed onto a surface of the complex through interaction between Ca²⁺ and tannic acid, rather than relying solely on electrostatic interaction of raw materials. Further, Ca²⁺ can form a network structure by combining with the carboxyl groups of carboxymethyl chitosan that better entraps protein nanoparticles therein.

In addition, there are amino groups in carboxymethyl chitosan, which are partially protonated and carry positive charges under acidic conditions. The protonated amino groups can bind to negatively charged regions of zein through electrostatic interaction, a stable zein-tannic acid-Ca²⁺-carboxymethyl chitosan structure is finally formed, thereby improving the tightness and strength of their binding and protecting the encapsulated quercetin therein. TA and Ca²⁺ can create a cross-linked network with complementary properties to form a strong and tough delivery system, thereby improving the stability of quercetin. The network formed by tannic acid cross-linked protein is soft and elastic, helping to maintain structural integrity during deformation. Furthermore, the network formed by calcium ion cross-linking is strong and can effectively dissipate energy, thus achieving purposes of reducing leakage and improving stability of quercetin during cooking. The method is simple, green, and highly feasible. After the dual cross-linked nanoparticles are cooked at 90° C. for 30 minutes, a retention rate of quercetin is increased by 28.6% compared with pure zein nanoparticles, and a retention rate of quercetin is increased by 20.6% compared with the prior art (adding a polysaccharide coating), and by 9.3% compared with single cross-linking (tannic acid cross-linking).

A first objective of the present disclosure is to provide a preparation method for polyphenol-ion dual cross-linked nanoparticles, including the following steps:
(1) dispersing zein in an ethanol solution, stirring to obtain a zein solution, and adjusting pH;
(2) dispersing tannic acid in an ethanol solution, stirring to obtain a tannic acid solution, and adjusting pH of the tannic acid solution;
(3) adding the tannic acid solution dropwise to the zein solution, stirring and mixing evenly, adjusting pH to make the solution fully exposed to oxygen for reaction, dialysis and drying;
(4) dissolving freeze-dried and cross-linked zein in an ethanol solution to obtain Solution A;
(5) adding quercetin to Solution A and stirring until completely dissolved to obtain Solution B;
(6) adding Solution B dropwise to a carboxymethyl chitosan solution and stirring thoroughly to obtain Solution C;
(7) adding a CaCl₂ solution to Solution C, stirring and mixing for reaction, and removing ethanol to obtain Solution D; and
(8) adjusting pH of Solution D, making up the evaporated ethanol with deionized water of the corresponding pH, and centrifuging to remove free quercetin.

In one embodiment of the present disclosure, a concentration of the ethanol solution in the step (1) is 75%-85%.

In one embodiment of the present disclosure, a concentration of the tannic acid in the step (2) is 0.6-4 mg/ml, the stirring in the step (2) lasts for 2-3 hours, and a rotation speed is 600-900 rpm; and
optionally, the concentration of the tannic acid is 2-4 mg/ml.

In one embodiment of the present disclosure, the pH in the step (2) refers to adjusting the pH to 9-12, and the reaction lasts for 20-24 hours; and a volume fraction of the ethanol solution in the step (2) is 75%-85%.

In one embodiment of the present disclosure, a volume ratio of the zein solution to the tannic acid solution in the step (3) is 0.8-1.2:0.8-1.2; and
optionally, the volume ratio is 1:1.

In one embodiment of the present disclosure, deionized water is replaced every 4 to 6 hours during the dialysis in the step (3), the temperature ranges from 10° C.-25° C., and the dialysis lasts from 24-48 hours.

In one embodiment of the present disclosure, the drying method in the step (2) is vacuum freeze drying.

In one embodiment of the present disclosure, a volume fraction of the ethanol solution in the step (3) is 75%-85%; the rotation speed in the step (3) is 600-900 rpm; in the step (3), the pH is 9-12, and the reaction lasts for 20-24 hours; a dialysis bag for the dialysis in the step (3) has a molecular weight of 14000 Da; the dialysis lasts for 24-48 hours; and the drying in the step (3) is performed through vacuum freeze drying.

In one embodiment of the present disclosure, a volume fraction of the ethanol solution in the step (4) is 75%-85%.

In one embodiment of the present disclosure, the stirring in the step (4) lasts for 2-3 hours, and a rotation speed is 600-900 rpm.

In one embodiment of the present disclosure, the polyphenol in the step (5) is quercetin, and a concentration of the quercetin is 0.1 mg/ml-0.2 mg/ml; a ratio of use amounts of the tannic acid cross-linked zein and the polyphenol in the step (5) is 10:1-20:1; and preferably, the stirring in the step (5) lasts for 2-3 hours, and a rotation speed is 600-900 rpm.

In one embodiment of the present disclosure, a concentration of the carboxymethyl chitosan solution in the step (6) is 0.25 mg/ml-5 mg/ml. Preferably, a mass ratio of the carboxymethyl chitosan to the cross-linked zein in the step (6) is 1:10-2:1; and preferably, the stirring in the step (6) lasts for 1-1.5 hours, and a rotation speed is 800-900 rpm.

In one embodiment of the present disclosure, the carboxymethyl chitosan was added to deionized water and stirred at 600-900 rpm overnight until completely dissolved and hydrated to obtain 1.67 mg/ml carboxymethyl chitosan solution.

In one embodiment of the present disclosure, a concentration of the CaCl₂ in the step (7) is 4-12 mM, the rotation speed in the step (7) is 600-900 rpm, and the pH in the step (7) is 6-6.5; and the centrifugation in the step (8) is performed at 3000-4000 rpm for 5-15 minutes.

In one embodiment of the present disclosure, the method for removing the ethanol in the step (8) is rotary evaporation, and the rotary evaporation is performed at 35-45° C. at a rate of 40-60 rpm for 10-15 minutes.

The present disclosure further provides polyphenol-ion dual cross-linked nanoparticles prepared by the above method.

A second objective of the present disclosure is to provide a preparation method for improving the thermal stability of functional polyphenol.

A third objective of the present disclosure is to provide application of the dual cross-linked nanoparticles in cooking food.

The present disclosure further provides a method for improving the thermal stability of polyphenol, including the following steps:
(1) dispersing zein in an ethanol solution, stirring to obtain a zein solution, and adjusting pH;
(2) dispersing tannic acid in an ethanol solution, stirring to obtain a tannic acid-ethanol solution, and adjusting pH of the solution;
(3) adding the tannic acid solution dropwise to the zein solution obtained in the step (1), adjusting pH to make the solution fully exposed to oxygen, and mixing for reaction, dialysis and drying;
(4) stirring and dissolving the freeze-dried zein obtained in the step (3) in an ethanol solution to obtain Solution A;
(5) adding polyphenol to Solution A and stirring until completely dissolved to obtain Solution B;
(6) adding Solution B dropwise to a carboxymethyl chitosan solution and stirring thoroughly to obtain Solution C;
(7) adding a CaCl₂ solution to Solution C, stirring and mixing for reaction to obtain Solution D; and
(8) adjusting pH of Solution D, removing ethanol through rotary evaporation, making up the evaporated ethanol with deionized water of the corresponding pH, and centrifuging to remove free polyphenol.

In one embodiment of the present disclosure, a concentration of the zein solution in the step (1) is 20-40 mg/ml.

In one embodiment of the present disclosure, a volume fraction of the ethanol solution in the step (1) is 75%-85%, and the pH in the step (1) is 9-12.

In one embodiment of the present disclosure, the polyphenol includes but is not limited to quercetin.

In one embodiment of the present disclosure, a concentration of the tannic acid solution in the step (2) is 0.6-4 mg/ml.

In one embodiment of the present disclosure, a volume fraction of the ethanol solution in the step (2) is 75%-85%.

In one embodiment of the present disclosure, in the step (3), the pH is 9-12, and the reaction lasts for 20-24 hours.

In one embodiment of the present disclosure, a dialysis bag for the dialysis in the step (3) has a molecular weight of 14000 Da; and the dialysis lasts for 24-48 hours.

In one embodiment of the present disclosure, the drying in the step (3) is performed through vacuum freeze drying.

In one embodiment of the present disclosure, a ratio of use amounts of the tannic acid cross-linked zein and the polyphenol in the step (5) is 10:1-20:1.

In one embodiment of the present disclosure, the stirring in the step (5) lasts for 2-3 hours, and a rotation speed is 600-900 rpm.

In one embodiment of the present disclosure, a mass ratio of the carboxymethyl chitosan to the cross-linked zein in the step (6) is 1:10-2:1.

In one embodiment of the present disclosure, the stirring in the step (6) lasts for 1-1.5 hours, and a rotation speed is 800-900 rpm.

In one embodiment of the present disclosure, a concentration of the CaCl₂ solution in the step (7) is 4-12 mM.

In one embodiment of the present disclosure, the pH in the step (8) is 6-6.5.

In one embodiment of the present disclosure, the method for removing the ethanol in the step (9) is rotary evaporation, the rotary evaporation is performed at 35-45° C. at a rate of 40-60 rpm for 10-15 minutes.

In one embodiment of the present disclosure, the centrifugation in the step (8) is performed at 3000-4000 rpm for 5-15 minutes.

The present disclosure provides application of the zein-carboxymethyl chitosan nanoparticles or the method in preparing food and medical supplies.

In one embodiment of the present disclosure, a juice beverage is prepared using zein-carboxymethyl chitosan nanoparticles with the following steps: according to parts by weight, 3-5 parts of zein-carboxymethyl chitosan nanoparticles, 20-25 parts of juice, 5-10 parts of white sugar, 3-5 parts of xanthan gum, and water added to make up to 100 parts.

In one embodiment of the present disclosure, an oral anti-inflammatory drug is prepared using zein-carboxymethyl chitosan nanoparticles as follows: zein-carboxymethyl chitosan nanoparticles loaded with quercetin are used; according to parts by weight, 5-20 parts of zein-carboxymethyl chitosan nanoparticles loaded with quercetin, 5-10 parts of lactose, 5-10 parts of microcrystalline cellulose, and starch added to make up to 100 parts are mixed uniformly and compressed into tablets to obtain the oral anti-inflammatory drug.

### Beneficial Effects

(1) In the present disclosure, tannic acid is first used to chemically cross-link zein, Ca²⁺ is then used to cross-link the negatively charged tannic acid in the protein with the carboxyl groups of carboxymethyl chitosan. In addition, the electrostatic interaction between the negatively charged hydroxyl groups of tannic acid and the protonated amine groups in the carboxymethyl chitosan, as well as the hydrogen bonds and the hydrophobic interaction between the zein-tannic acid covalent complex and the carboxymethyl chitosan are used to prepare high-strength nanoparticles with a three-dimensional cross-linked network of protein-polyphenol-Ca²⁺-polysaccharide, so as to achieve the purpose of improving the stability of quercetin during thermal processing, thereby overcoming the defect that the existing delivery system cannot protect the internal quercetin due to its instability.

(2) When being applied in the steamed food, the dual cross-linked nanoparticles with thermal stability in the present disclosure can increase the retention rate of quercetin by 28.6% compared with pure zein nanoparticles, and increase by 20.6% compared with the prior art (adding a coating), and by 9.3% compared with single cross-linking (tannic acid cross-linking), thereby significantly expanding the application scope of quercetin in food processing.

### BRIEF DESCRIPTION OF THE DRAWINGS

FIG. 1 is a schematic principle diagram according to the present disclosure.

FIG. 2 shows retention rates of quercetin in nanoparticles obtained in Examples 1-7 and Comparative Examples 1-7 according to the present disclosure after cooking at 90° C. for 30 minutes.

FIG. 3 shows thermogravimetric analysis results of nanoparticles obtained in Examples 1-7 and Comparative Examples 1-7 according to the present disclosure.

FIG. 4 is a Fourier transform infrared spectrometer image of raw material of dual cross-linked nanoparticles obtained in Example 4 according to the present disclosure.

FIG. 5. is a scanning electron microscope image of dual cross-linked nanoparticles obtained in Example 4 according to the present disclosure.

### DETAILED DESCRIPTIONS OF THE EMBODIMENTS

The present disclosure will be described in further detail below in conjunction with specific embodiments, but the implementation modes of the present disclosure are not limited thereto.

The detection methods used in the following examples are as follows:

### Determination of Retention Rate after Heating

10 mL of a sample under test is placed in a test tube and incubating in a water bath at 90° C. for 30 minutes; the sample under test before and after heating is centrifuged at 10,000 rpm for 10 minutes, collecting supernatant and diluting the supernatant with an ethanol solution; a content of quercetin in the ethanol solution is measured at a wavelength of 374 nm using a UV-Vis spectrophotometer (UV-5200, Metash, China); and a suitable calibration curve is measured to calculate a retention rate of the quercetin, and a retention rate after heating is calculated according to the following formula.

$$Retention rate ( \% ) = \frac{Content of quercetin after heating}{Content of quercetin before heating} \times 100 \% \qquad ( 1 )$$

### Analysis of Thermal Stability

2-3 mg of the sample is weighed and placed in a crucible, which is heated from 30° C. to 600° C. at a constant rate of 10° C./minute, with a nitrogen flow rate controlled at 20 mL/minute; and a thermogravimetric curve is plotted according to the obtained data.

### Determination of Particle Size

Particle sizes of nanoparticles are measured using a Malvern particle size and Zeta-potential analyzer (Malvern Instruments Ltd.).

### Infrared Spectrum Test

A Fourier transform infrared (FTIR) spectrum of the sample is measured using an FTIR spectrometer, the sample is mixed with KBr powder, ground and pressed to prepare into a thin slice, which is then placed in the FTIR spectrometer for analysis, with a wavenumber range of 400-4000 cm⁻¹.

### Scanning Electron Microscopy

The sample is diluted 10 times with deionized water at pH 6 and dropped onto a silicon wafer for natural air drying; and the silicon wafer is attached to conductive adhesive, sprayed with gold, and then observed at magnifications of 30k and 60k, and photographed.

### EXAMPLE 1: INFLUENCE OF TANNIC ACID AND CACL₂ DUAL CROSS-LINKED ZEIN-CARBOXYMETHYL CHITOSAN NANOPARTICLES ON THERMAL STABILITY OF QUERCETIN

Specific steps were as follows:
(1) zein was dispersed in 75% ethanol solution and stirred at 600 rpm for 2 hours to obtain 40 mg/ml zein ethanol solution, and a pH value was adjusted to 9;
(2) tannic acid was added to 75% ethanol solution and stirred at 600 rpm for 2 hours to obtain a tannic acid ethanol solution a final concentration of 2 mg/ml, and a pH value was adjusted to 9;
(3) the tannic acid ethanol solution obtained in the step (2) was added dropwise to the zein ethanol solution obtained in the step (1) (at a volume ratio of 1:1) to obtain a solution, a pH value was adjusted to 9, the solution was added in a brown bottle (sealed with tin foil and punctured with a syringe on a surface of the tin foil), and then placed in a magnetic stirrer for stirring at 500 rpm to ensure sufficient contact with oxygen (exposed to air) for further reaction for 24 hours, the resulting solution was transferred into a dialysis device, the dialysis device was placed in deionized water for 48 hours (to obtain dialysate with a molecular weight ranging from 25,000 Da-45,000 Da); and the dialysate was freeze-dried to obtain tannic acid covalently cross-linked zein;
(4) the freeze-dried tannic acid covalently cross-linked zein was dissolved in 75% ethanol and stirred at 600 rpm for 3 hours until completely dissolved to obtain 10 mg/ml tannic acid covalently cross-linked zein solution;
(5) quercetin was added to the tannic acid covalently cross-linked zein solution obtained in the step (4) and stirred at 600 rpm for 3 hours until completely dissolved to obtain a solution, where a concentration of the quercetin was 0.5 mg/ml;
(6) carboxymethyl chitosan was added to deionized water and stirred at 900 rpm overnight until completely dissolved and hydrated to obtain 1.67 mg/ml carboxymethyl chitosan solution;
(7) the solution obtained in the step (5) was added dropwise to the carboxymethyl chitosan solution obtained in the step (6) (at a volume ratio 1:3) to obtain a solution, and the solution was stirred at 900 rpm for 1 hour to obtain a dispersion; and
(8) CaCl₂ solution (4 mM/ml) was added to the solution obtained in the step (7) to make a concentration of CaCl₂ in a final system up to 0.2 mM/ml, the solution was rapidly stirred and mixed, a pH value was adjusted to 6, ethanol was removed through rotary evaporation (40° C., −0.01 Mpa), deionized water was added to make up the evaporated ethanol, and free quercetin was removed by centrifugation (3000 rpm, 10 minutes).

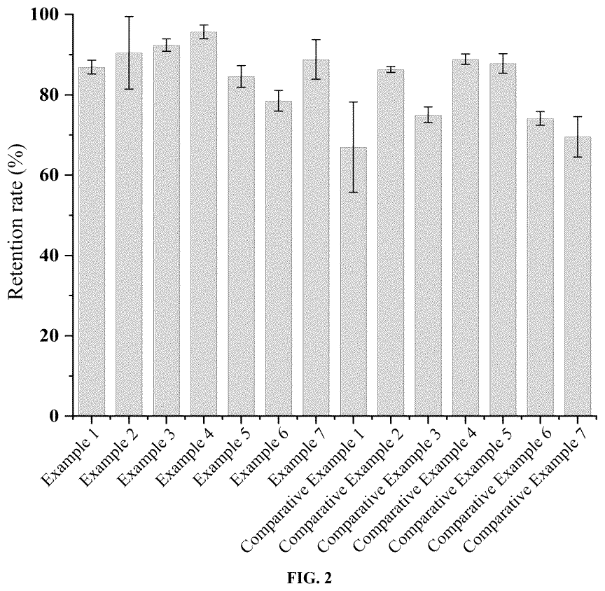

A retention rate of quercetin in the dual cross-linked nanoparticles prepared in this example was shown in FIG. 2.

### EXAMPLE 2

Specific implementation mode was the same as that in Example 1, except that a concentration of CaCl₂ added in the step (8) in Example 1 was 6 mM/ml to make a concentration of CaCl₂ in a final system up to 0.3 mM/ml, and other steps were consistent with those in Example 1 to obtain dual cross-linked nanoparticles capable of improving the thermal stability of quercetin.

### EXAMPLE 3

Specific implementation mode was the same as that in Example 1, except that a concentration of CaCl₂ added in the step (8) in Example 1 was 8 mM/ml to make a concentration of CaCl₂ in a final system up to 0.4 mM/ml, and other steps were consistent with those in Example 1 to obtain dual cross-linked nanoparticles capable of improving the thermal stability of quercetin.

### EXAMPLE 4

Specific implementation mode was the same as that in Example 1, except that a concentration of CaCl₂ added in the step (8) in Example 1 was 10 mM/ml to make a concentration of CaCl₂ in a final system up to 0.5 mM/ml, and other steps were consistent with those in Example 1 to obtain dual cross-linked nanoparticles capable of improving the thermal stability of quercetin.

### EXAMPLE 5

Specific implementation mode was the same as that in Example 1, except that a concentration of CaCl₂ added in the step (8) in Example 1 was 12 mM/ml to make a concentration of CaCl₂ in a final system up to 0.6 mM/ml, and other steps were consistent with those in Example 1 to obtain dual cross-linked nanoparticles capable of improving the thermal stability of quercetin.

### EXAMPLE 6

Specific implementation mode was the same as that in Example 1, except that a concentration of the tannic acid ethanol solution in the step (2) in Example 1 was 0.67 mg/ml, and other steps were consistent with those in Example 1 to obtain dual cross-linked nanoparticles capable of improving the thermal stability of quercetin.

### EXAMPLE 7

Specific implementation mode was the same as that in Example 1, except that a concentration of the tannic acid ethanol solution in the step (2) in Example 1 was 4 mg/ml, and other steps were consistent with those in Example 1 to obtain dual cross-linked nanoparticles capable of improving the thermal stability of quercetin.

### COMPARATIVE EXAMPLE 1: INFLUENCE OF PURE ZEIN ON THERMAL STABILITY OF QUERCETIN

Specific steps were as follows:
(1) zein was dispersed in 75% ethanol solution and stirred at 600 rpm for 3 hours to obtain 10 mg/ml zein ethanol solution;
(2) quercetin was added to the zein solution and stirred at 600 rpm for 1 hour until completely dissolved to obtain a solution, where a concentration of the quercetin was 0.5 mg/ml; and
(3) the solution obtained in the step (2) was added dropwise to deionized water at a volume ratio of 1:3, and then stirred at 900 rpm for 1 hour, ethanol was removed through rotary evaporation (40° C., −0.01 Mpa), deionized water was added to make up the evaporated ethanol, and free quercetin was removed by centrifugation (3000 rpm, 10 minutes).

### COMPARATIVE EXAMPLE 2: INFLUENCE OF TANNIC ACID COVALENTLY CROSS-LINKED ZEIN ON THERMAL STABILITY OF QUERCETIN

Specific steps were as follows:
(1) zein was dispersed in 75% ethanol solution and stirred at 600 rpm for 2 hours to obtain 40 mg/ml zein ethanol solution, and a pH value was adjusted to 9;
(2) tannic acid was added to 75% ethanol solution and stirred at 600 rpm for 2 hours to obtain a tannic acid ethanol solution a final concentration of 2 mg/ml, and a pH value was adjusted to 9;
(3) the tannic acid ethanol solution obtained in the step (2) was added dropwise to the zein ethanol solution obtained in the step (1) to obtain a solution, a pH value was adjusted to 9, the solution was added in a brown bottle (sealed with tin foil and punctured with a syringe on a surface of the tin foil), and then placed in a magnetic stirrer for stirring at 500 rpm to ensure sufficient contact with oxygen for further reaction for 24 hours, the resulting solution was transferred into a dialysis device, the dialysis device was placed in deionized water for 48 hours (to obtain dialysate with a molecular weight ranging from 25,000 Da to 45,000 Da); and the dialysate was frozen and dried to obtain tannic acid covalently cross-linked zein.
(4) the freeze-dried tannic acid covalently cross-linked zein was dissolved in 75% ethanol solution and stirred at 600 rpm for 3 hours until completely dissolved to obtain 10 mg/ml tannic acid covalently cross-linked zein solution;
(5) quercetin was added to the tannic acid covalently cross-linked zein solution obtained in the step (4) and stirred at 600 rpm for 1 hours until completely dissolved to obtain a solution, where a concentration of the quercetin was 0.5 mg/ml; and
(6) the solution obtained in the step (5) was added dropwise to deionized water at a volume ratio of 1:3, and then stirred at 900 rpm for 1 hour, ethanol was removed through rotary evaporation (40° C., −0.01 Mpa), deionized water was added to make up the evaporated ethanol, and free quercetin was removed by centrifugation (3000 rpm, 10 minutes).

### COMPARATIVE EXAMPLE 3: INFLUENCE OF ZEIN-CARBOXYMETHYL CHITOSAN NANOPARTICLES ON THERMAL STABILITY OF QUERCETIN

Specific steps were as follows:
(1) zein was dispersed in 75% ethanol solution and stirred at 600 rpm for 3 hours to obtain 10 mg/ml zein ethanol solution;
(2) quercetin was added to the zein ethanol solution and stirred at 600 rpm for 1 hour until completely dissolved to obtain a solution, where a concentration of the quercetin was 0.5 mg/ml;
(3) carboxymethyl chitosan was added to deionized water and stirred at 900 rpm overnight until completely dissolved to obtain 1.67 mg/ml carboxymethyl chitosan solution;
(4) the solution obtained in the step (2) was added dropwise to the 1.67 mg/ml carboxymethyl chitosan solution obtained in the step (3) at a volume ratio of 1:3, and then stirred at 900 rpm for 1 hour, a pH value was adjusted to 6, ethanol was removed through rotary evaporation (40° C., −0.01 Mpa), deionized water (pH 6) was added to make up the evaporated ethanol, and free quercetin was removed by centrifugation (3000 rpm, 10 minutes).

### COMPARATIVE EXAMPLE 4: EFFECT OF TANNIC ACID COVALENTLY CROSS-LINKED ZEIN-CARBOXYMETHYL CHITOSAN NANOPARTICLES ON THERMAL STABILITY OF QUERCETIN

Specific steps were as follows:
(1) zein was dispersed in 75% ethanol solution and stirred at 600 rpm for 2 hours to obtain 40 mg/ml zein ethanol solution, and a pH value was adjusted to 9;
(2) tannic acid was added to 75% ethanol solution and stirred at 600 rpm for 2 hours to obtain tannic acid ethanol solution a final concentration of 2 mg/ml, and a pH value was adjusted to 9;
(3) the tannic acid ethanol solution obtained in the step (2) was added dropwise to the zein ethanol solution obtained in the step (1) to obtain a solution, a pH value was adjusted to 9, the solution was added in a brown bottle (sealed with tin foil and punctured with a syringe on a surface of the tin foil), and then placed in a magnetic stirrer for stirring at 500 rpm to ensure sufficient contact with oxygen for further reaction for 24 hours, the resulting solution was transferred into a dialysis device, the dialysis device was placed in deionized water for 48 hours (to obtain dialysate with a molecular weight ranging from 25,000 Da-45,000 Da); and the dialysate was frozen and dried to obtain tannic acid covalently cross-linked zein;
(4) the freeze-dried tannic acid covalently cross-linked zein was dissolved in 75% ethanol solution and stirred at 600 rpm for 3 hours until completely dissolved to obtain 10 mg/ml tannic acid covalently cross-linked zein solution;
(5) quercetin was added to the tannic acid covalently cross-linked zein solution obtained in the step (4) and stirred at 600 rpm for 2 hours until completely dissolved to obtain a solution, where a concentration of the quercetin was 0.5 mg/ml;
(6) carboxymethyl chitosan was added to deionized water and stirred at 900 rpm overnight until completely dissolved to obtain 1.67 mg/ml carboxymethyl chitosan solution;
(7) the solution obtained in the step (5) was added dropwise to the carboxymethyl chitosan solution obtained in the step (6) at a volume ratio of 1:3, and then stirred at 900 rpm for 1 hour, a pH value was adjusted to 6, ethanol was removed through rotary evaporation (40° C., −0.01 Mpa), and deionized water was added to make up the evaporated ethanol.

### COMPARATIVE EXAMPLE 5: INFLUENCE OF GENIPIN AND CACL₂ DUAL CROSS-LINKED ZEIN-CARBOXYMETHYL CHITOSAN NANOPARTICLES ON THERMAL STABILITY OF QUERCETIN

(1) Zein was dispersed in 75% ethanol solution and stirred at 600 rpm for 3 hours to obtain 10 mg/ml zein ethanol solution;
(2) quercetin was added to the zein ethanol solution and stirred at 600 rpm for 1 hour until completely dissolved to obtain a solution, where a concentration of the quercetin was 0.5 mg/ml;
(3) carboxymethyl chitosan was added to deionized water and stirred at 900 rpm overnight until completely dissolved to obtain 1.67 mg/ml carboxymethyl chitosan solution;
(4) the solution obtained in the step (2) was added dropwise to the carboxymethyl chitosan solution obtained in the step (3) at a volume ratio of 1:3, and then stirred at 900 rpm for 1 hour, a pH value was adjusted to 6, and ethanol was removed through rotary evaporation (40° C., −0.01 Mpa) to obtain a dispersion;
(5) 2 ml genipin solution (5 mmol/L) was added to the dispersion obtained in the step (4) and stirred at 900 rpm to obtain genipin cross-linked zein-carboxymethyl chitosan nanoparticles loaded with the quercetin; and
(6) CaCl₂ solution was added to the nanoparticle dispersion obtained in the step (5) to make a concentration of CaCl₂ in a final system up to 0.4 mM/ml, the dispersion was rapidly stirred at 900 rpm, ethanol was removed through rotary evaporation (40° C., −0.01 Mpa), deionized water was added to make up the evaporated ethanol and solution, free quercetin was removed by centrifugation (3000 rpm, 10 minutes) to obtain the genipin and CaCl₂ dual cross-linked zein-carboxymethyl chitosan nanoparticles loaded with the quercetin.

### COMPARATIVE EXAMPLE 6: INFLUENCE OF KCL AND CACL₂ DUAL CROSS-LINKED ZEIN-CARBOXYMETHYL CHITOSAN NANOPARTICLES ON THERMAL STABILITY OF QUERCETIN

(1) Zein was dispersed in 75% ethanol solution and stirred at 600 rpm for 3 hours to obtain 10 mg/ml zein ethanol solution;
(2) quercetin was added to the zein ethanol solution and stirred at 600 rpm for 1 hour until completely dissolved to obtain a solution, where a concentration of the quercetin was 0.5 mg/ml;
(3) carboxymethyl chitosan was added to deionized water and stirred at 900 rpm overnight until completely dissolved to obtain 1.67 mg/ml carboxymethyl chitosan solution;
(4) the solution obtained in the step (2) was added dropwise to the carboxymethyl chitosan solution obtained in the step (3) (at a volume ratio 1:3) to obtain a solution, and the solution was stirred at 900 rpm for 1 hour to obtain a dispersion; and

(5) KCl solution (4 mM/ml) and CaCl₂ solution (4 mM/ml) were added to the dispersion obtained in the step (4) to make concentrations of KCl and CaCl₂ in a final system both up to 0.2 mM/ml, the dispersion was rapidly stirred and mixed, a pH value was adjusted to 6, ethanol was removed through rotary evaporation (40° C., −0.01 Mpa), deionized water was added to make up the evaporated ethanol, and free quercetin was removed by centrifugation (3000 rpm, 10 minutes) to obtain the KCl and CaCl₂ dual cross-linked zein-carboxymethyl chitosan nanoparticles.

### COMPARATIVE EXAMPLE 7: INFLUENCE OF GLUTAMYLTRANSFERASE AND CACL₂ DUAL CROSS-LINKED ZEIN-CARBOXYMETHYL CHITOSAN NANOPARTICLES ON THERMAL STABILITY OF QUERCETIN

(1) Zein was dispersed in 75% ethanol solution and stirred at 600 rpm for 3 hours to obtain 10 mg/ml zein ethanol solution;
(2) quercetin was added to the zein ethanol solution and stirred at 600 rpm for 1 hour until completely dissolved to obtain a solution, where a concentration of the quercetin was 0.5 mg/ml;
(3) carboxymethyl chitosan was added to deionized water and stirred at 900 rpm overnight until completely dissolved to obtain 1.67 mg/ml carboxymethyl chitosan solution;
(4) the solution obtained in the step (2) was added dropwise to the carboxymethyl chitosan solution obtained in the step (3) at a volume ratio of 1:3, and then stirred at 900 rpm for 1 hour, a pH value was adjusted to 6, and ethanol was removed through rotary evaporation (40° C., −0.01 Mpa) to obtain a dispersion;
(5) 30 U/g glutamyltransferase was added to the dispersion obtained in the step (4) and reacted in a water bath at a constant temperature of 50° C. for 60 minutes, then treated at 75° C. for 15 minutes to inactivate enzyme, and the dispersion was stirred at 600 rpm to obtain glutamyltransferase-cross-linked quercetin-loaded zein-carboxymethyl chitosan nanoparticles loaded with the quercetin; and
(6) CaCl₂ solution was added to the nanoparticle dispersion obtained in the step (5) to make a concentration of CaCl₂ in a final system up to 0.4 mM/ml, the dispersion was rapidly stirred at 900 rpm, ethanol was removed through rotary evaporation, deionized water was added to make up the evaporated ethanol and solution, free quercetin was removed by centrifugation (3000 rpm, 10 minutes) to obtain the glutamyltransferase and CaCl₂ dual cross-linked zein-carboxymethyl chitosan nanoparticles loaded with the quercetin.

### EXPERIMENTAL RESULTS

The nanoparticles loaded with the polyphenol obtained in Examples 1-7 and Comparative Examples 1-7 were subjected to performance test, and test results were shown in Table 1, FIGS. 2-4.

### 1. Particle Size Test Results

TABLE 1
Particle size test results of nanoparticles obtained
in Examples 1-7 and Comparative Examples 1-7

| Sample | Particle size (nm) |
|---|---|
| Comparative Example 1 | 248.00 ± 3.07 |
| Comparative Example 2 | 136.67 ± 11.30 |
| Comparative Example 3 | 315.33 ± 37.91 |
| Comparative Example 4 | 199.80 ± 3.12 |
| Comparative Example 5 | 319.23 ± 7.57 |
| Comparative Example 6 | 230.30 ± 11.23 |
| Comparative Example 7 | 232.00 ± 3.32 |
| Example 1 | 274.00 ± 20.07 |
| Example 2 | 245.37 ± 1.02 |
| Example 3 | 224.40 ± 2.25 |
| Example 4 | 221.73 ± 1.91 |
| Example 5 | 218.57 ± 4.04 |
| Example 6 | 255.80 ± 17.68 |
| Example 7 | 621.83 ± 24.63 |

As can be seen from Table 1, the particle size of nanoparticles obtained in Comparative Example 2 was smaller than that of Comparative Example 1, indicating that the tannic acid cross-linked with the zein molecules could reach tighter binding. The particle size of nanoparticles obtained in Comparative Example 4 was smaller than that of Comparative Example 3, indicating that compared with the zein, the zein-tannic acid had stronger hydrogen bonds, electrostatic interaction, and hydrophobic interaction with the carboxymethyl chitosan. Examples 1-7 could generate nanoparticles, and the particle sizes of nanoparticles obtained in Examples 1-6 ranged from 200-300 nm. The particle size of nanoparticles obtained in Example 7 was 621.83 nm, forming relatively larger particles. As the concentration of CaCl₂ increased, the particle size of nanoparticles gradually decreased, indicating that the network structure between the carboxymethyl chitosan and the zein-tannic acid formed by cross-linking tannic acid and Ca²⁺ could make the nanoparticles tighter, thereby preventing the encapsulated quercetin from escaping.

### 2. Test of Thermal Stability

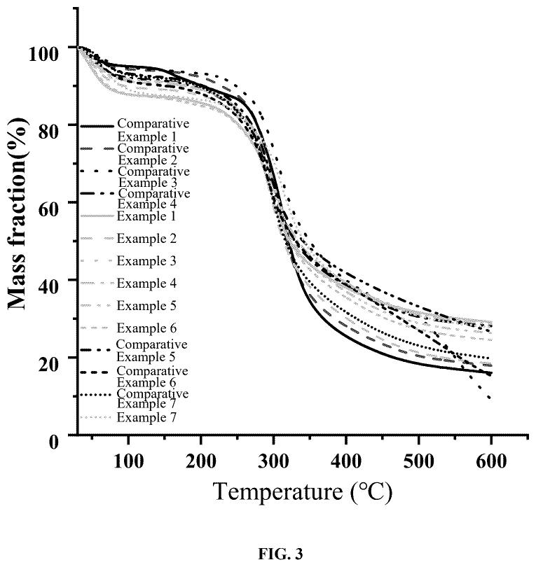

Table 2 shows the retention rates of quercetin after heating and the thermogravimetric analysis results of nanoparticles from Comparative Examples 1-7 and Examples 1-7, as shown in Table 2 and FIG. 3.

TABLE 2
Retention rate and thermogravimetric analysis results

| | Retention | Mass fraction of |
|---|---|---|
| Sample | rate (%) | final residue (%) |
| Comparative Example 1 | 66.98 ± 11.2 | 16.05 |
| Comparative Example 2 | 86.31 ± 2.01 | 17.89 |
| Comparative Example 3 | 75.03 ± 0.73 | 9.13 |
| Comparative Example 4 | 88.87 ± 1.32 | 28.10 |
| Comparative Example 5 | 87.80 ± 2.45 | 26.67 |
| Comparative Example 6 | 74.12 ± 1.76 | 15.22 |
| Comparative Example 7 | 69.54 ± 5.08 | 19.74 |
| Example 1 | 86.91 ± 1.72 | 29.11 |
| Example 2 | 90.44 ± 9.00 | 18.47 |
| Example 3 | 92.37 ± 1.55 | 29.14 |
| Example 4 | 95.65 ± 1.74 | 28.64 |
| Example 5 | 84.59 ± 2.70 | 26.31 |
| Example 6 | 78.53 ± 2.57 | 24.53 |
| Example 7 | 88.80 ± 4.92 | 27.8 |

As can be seen from the table, the retention rates of quercetin after heating of the dual cross-linked nanoparticles in Examples 2, 3, and 4 were 90.44%, 92.37%, and 95.65%, respectively, higher than those of all Comparative Examples, and the retention rate of quercetin in Example 4 was higher than those of Examples 2 and 3. Compared with Comparative Examples 1-3, the retention rates and mass fraction of final residue (the mass fraction of final residue after burning) in Examples 3 and 4 were significantly higher than those in Comparative Examples 1-3, indicating that the protective effects of the tannic acid and CaCl₂ dual cross-linked nanoparticles for quercetin was higher than that of the uncross-linked nanoparticles. Compared with Comparative Examples 5-7, the retention rates of quercetin in Examples 3 and 4 were higher than those in Comparative Examples 5-7, indicating that the protection effects of the tannic acid and CaCl₂ dual cross-linked nanoparticles for quercetin were greater than that of other dual cross-linking methods. Compared with Comparative Example 4, the retention rates of quercetin in Examples 3 and 4 were higher, indicating that the protection effects of the tannic acid and CaCl₂ dual cross-linked nanoparticles for quercetin are greater than the tannic acid cross-linked nanoparticles. The thermogravimetric analysis showed that the mass fraction of residue in Examples 3 and 4 (the mass fraction of final residue after burning) were 29.14% and 28.64%, respectively, higher than those of all the Comparative Examples. To sum up, Example 4 provided the best protection effects for quercetin and exhibited the best thermal stability.

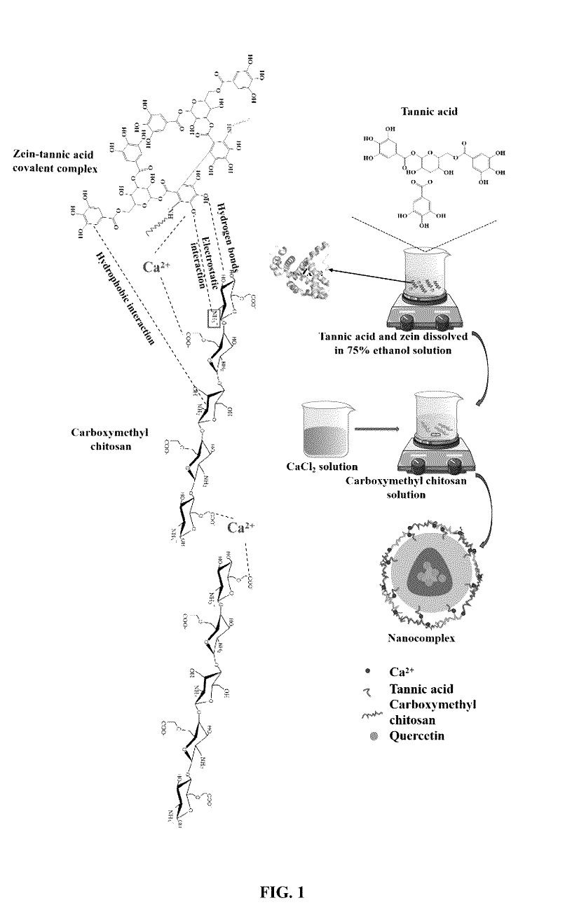

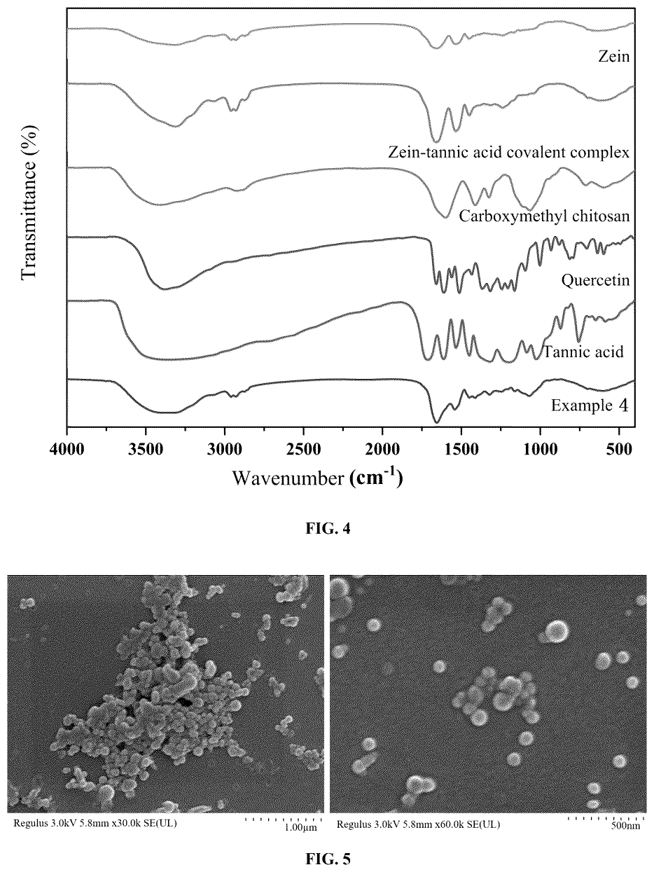

FIG. 4 is an FTIR spectrum of the dual cross-linked nanoparticles obtained in Example 4. As can be seen from FIG. 1, peaks observed in ranges of 1530-1660 cm⁻¹ and 1400-1451 cm⁻¹ for carboxymethyl chitosan are likely related to the vibration of carboxylic acid anions. The characteristic peaks of zein were observed at 1657.92 cm⁻¹ (amide I) and 1536.82 cm⁻¹ (amide II, —C—N and —N—H). The peaks of the zein-tannic acid covalent complex obtained after cross-linking with the tannic acid changed at amide I and amide II, indicating that a covalent bond formed between the amino groups of zein and tannic acid. Finally, Example 4, the sharp characteristic peaks of quercetin were not observed in the spectrum range of 750-1750 cm⁻¹ in Example 4, this was because the quercetin was encapsulated in the dual cross-linked nanoparticles in an amorphous form. The results indicated that hydrogen bonding, amide bonds, hydrophobic interactions, and electrostatic interactions were all involved in the formation of the dual cross-linked nanoparticles.

FIG. 5 is a scanning electron microscope image of the dual cross-linked nanoparticles obtained in Example 4 at different magnifications. As can be seen from the image, a great number of carboxymethyl chitosan were distributed on a surface of the nanoparticles, and the nanoparticles had a spherical structure, with a particle size of about 200 nm.

Although the present disclosure has been disclosed as above in the form of preferred embodiments, it is not intended to limit the present disclosure. Those skilled in the art can make various modifications and variations without departing from the spirit and scope of the present disclosure. Therefore, the scope of protection of the present disclosure should be defined by the claims.

1. A dual cross-linked zein-carboxymethyl chitosan nanoparticle for improving thermal stability of quercetin, wherein a preparation method for the zein-carboxymethyl chitosan nanoparticle comprises the following steps:
(1) dispersing zein in an ethanol solution, stirring to obtain a zein solution with a zein concentration of 40 mg/mL, and adjusting pH of the zein solution to 9-12;
(2) dispersing tannic acid in an ethanol solution, stirring to obtain a tannic acid-ethanol solution with a tannic acid concentration of 2 mg/mL, and adjusting pH of the tannic acid-ethanol solution;
(3) adding the tannic acid-ethanol solution dropwise to the zein solution obtained in the step (1), adjusting pH to make the solution fully exposed to oxygen, and mixing for reaction, dialysis and drying to obtain zein;
(4) dissolving the dried zein obtained in the step (3) in an ethanol solution to obtain a solution A with a tannic acid-zein covalent complex concentration of 10 mg/mL;
(5) adding quercetin to the solution A and stirring until completely dissolved to obtain a solution B with a quercetin concentration of 0.5 mg/mL;
(6) adding the solution B dropwise to a carboxymethyl chitosan solution with a carboxymethyl chitosan concentration of 1.67 mg/mL at a volume ratio of 1:3, and stirring thoroughly to obtain a solution C;
(7) adding a CaCl₂ solution to the solution C, stirring and mixing for reaction to obtain a solution D with a CaCl₂ concentration of 0.5 mM; and
(8) adjusting pH of the solution D, removing ethanol through rotary evaporation, making up the evaporated ethanol with deionized water of the corresponding pH, and centrifuging to remove free polyphenol to obtain the dual cross-linked zein-carboxymethyl chitosan nanoparticle for improving thermal stability of quercetin.

2. The zein-carboxymethyl chitosan nanoparticle according to claim 1, wherein a volume fraction of the ethanol solution in the step (1) is 75%-85%.

3. The zein-carboxymethyl chitosan nanoparticle according to claim 1 or 2, wherein a volume fraction of the ethanol solution in the step (2) is 75%-85%; the pH in the step (3) is 9-12, and the reaction lasts for 20-24 hours; a dialysis bag used for the dialysis in the step (3) has a molecular weight of 14000 Da; the dialysis lasts for 24-48 hours; and a method for the drying in the step (3) is vacuum freeze drying.

4. The zein-carboxymethyl chitosan nanoparticle according to claim 1 or 2, wherein the stirring in the step (5) lasts for 2-3 hours at a rotation speed of 600-900 rpm; and the stirring in the step (6) lasts for 1-1.5 hours at a rotation speed of 800-900 rpm.

5. The zein-carboxymethyl chitosan nanoparticle according to claim 1 or 2, wherein the pH in the step (8) is adjusted to 6-6.5; a method for removing the ethanol in the step (8) is rotary evaporation, and the rotary evaporation is performed at a temperature of 35-45° C. at a rate of 40-60 rpm for 10-15 minutes; and the centrifugation in the step (8) is performed at 3000-4000 rpm for 5-15 minutes.

6. A method for improving thermal stability of quercetin, comprising the following steps:
(1) dispersing zein in an ethanol solution, stirring to obtain a zein solution with a zein concentration of 20-40 mg/mL, and adjusting pH of the zein solution to 9-12;
(2) dispersing tannic acid in an ethanol solution, stirring to obtain a tannic acid-ethanol solution with a tannic acid concentration of 2 mg/mL, and adjusting pH of the tannic acid-ethanol solution;
(3) adding the tannic acid-ethanol solution dropwise to the zein solution obtained in the step (1), adjusting pH to make the solution fully exposed to oxygen, and mixing for reaction, dialysis and drying to obtain zein;
(4) dissolving the dried zein obtained in the step (3) in an ethanol solution to obtain a solution A with a tannic acid-zein covalent complex concentration of 10 mg/mL;
(5) adding quercetin to the solution A and stirring until completely dissolved to obtain a solution B with a quercetin concentration of 0.5 mg/mL;
(6) adding the solution B dropwise to a carboxymethyl chitosan solution with a concentration of 1.67 mg/mL at a volume ratio of 1:3, and stirring thoroughly to obtain a solution C;
(7) adding a CaCl₂ solution to the solution C, stirring and mixing for reaction to obtain a solution D with a CaCl₂ concentration of 0.5 mM; and
(8) adjusting pH of the solution D, removing ethanol through rotary evaporation, making up the evaporated ethanol with deionized water of the corresponding pH, and centrifuging to remove free polyphenol.

7. The method according to claim 6, wherein a volume fraction of the ethanol solution in the step (1) is 75%-85%; a volume fraction of the ethanol solution in the step (2) is 75%-85%; the pH in the step (3) is 9-12, and the reaction lasts for 20-24 hours; a dialysis bag used for the dialysis in the step (3) has a molecular weight of 14000 Da; the dialysis lasts for 24-48 hours; and a method for the drying in the step (3) is vacuum freeze drying.

8. The method according to claim 7, wherein the stirring in the step (5) lasts for 2-3 hours at a rotation speed of 600-900 rpm; and the stirring in the step (6) lasts for 1-1.5 hours at a rotation speed of 800-900 rpm.

9. The method according to claim 8, wherein the pH in the step (8) is adjusted to 6-6.5; a method for removing the ethanol in the step (8) is rotary evaporation, and the rotary evaporation is performed at a temperature of 35-45° C. at a rate of 40-60 rpm for 10-15 minutes; and the centrifugation in the step (8) is performed at 3000-4000 rpm for 5-15 minutes.

10. The zein-carboxymethyl chitosan nanoparticle according to any one of claims 1-5 or the method according to any one of claims 6-9 in preparing food and pharmaceutical products.
